(19) United States
(12) Patent Application Publication
(10) Pub. No.: US 2017/0029886 A1
(43) Pub. Date: Feb. 2, 2017

(54) HIGH-RESOLUTION HLA TYPING

(71) Applicant: ETABLISSEMENT FRANCAIS DU SANG, La Plaine Saint Denis Cedex (FR)

(72) Inventors: MEHDI ALIZADEH, RENNES (FR); GILBERT SEMANA, RENNES (FR)

(73) Assignee: Etablissement Francais Du Sang, La Plaine Saint Denis Cedex (FR)

(21) Appl. No.: 15/114,081

(22) PCT Filed: Feb. 17, 2015
(86) PCT No.: PCT/EP2015/053344

§ 371 Date: Jul. 26, 2016

(30) Foreign Application Priority Data
Feb. 18, 2014 (EP) 14305226.4

(51) Int. Cl. C12Q 1/68

(52) U.S. Cl. CPC C12Q 1/6881; C12Q 2600/172; C12Q 2600/16

(57) ABSTRACT

The present invention relates to improved methods and kits for typing HLA class I and class II loci using DNA amplification and sequencing.

### FIELD OF THE INVENTION

The present invention relates to methods and kits for high-resolution typing HLA genes.

### BACKGROUND OF THE INVENTION

The human leukocyte antigen (HLA) system is one of the most polymorphic regions of the human genome. To date, more than 10533 alleles have been identified for the HLA class I and class II loci, and this number is growing. Even if this system is one of the most extensively studied regions, this level of polymorphism remains a challenge when it comes to typing HLA genes.

HLA typing is routinely performed in connection with many medical indications such as transplantation, studies of HLA-related diseases or individual identification. In particular, in the case of transplantation, high-resolution HLA typing is required to accomplish the best possible histocompatibility between donor and recipient and thus to decrease the risk of graft-versus-host disease and mortality.

Currently, some of the widely used molecular methods for typing classical HLA class I and class II loci (HLA-A, -B, -C, DR, -DQ and -DP), are polymerase chain reaction (PCR) typing methods using sequence-specific oligonucleotide hybridization (SSO) or sequence-specific priming (SSP) and sequence-based typing (SBT). However, even if these methods provide high-resolution typing, genotyping ambiguity remains an issue in a lot of cases. Ambiguous allele assignments are produced either due to failure to interrogate all polymorphic positions, or when two or more different allele combinations produce identical sequences (cis/trans ambiguities). Resolving these ambiguities requires costly and laborious approaches due to the large and rapidly growing number of described HLA alleles.

Recently, new HLA typing strategies using next-generation sequencing (NGS) have been developed. In comparison to conventional HLA typing methods, these NGS-based techniques significantly reduce genotyping ambiguity (Danzer et al., BMC Genomics. 2013 Apr. 4; 14:221). However, to completely solve the problem of phase ambiguity and detect all polymorphisms such as single-nucleotide polymorphisms (SNPs) or indels that could result in null alleles, it is necessary to amplify and sequence the entire HLA loci (Shiina et al., Tissue Antigens. 2012 October; 80(4):305-16) and, to date, the complexity of the process as well as the running costs prevent the use of these methods in clinical laboratories.

Therefore, there is a great need to develop new NGS-based HLA-typing strategies that are simpler, faster and more cost effective and can thus be routinely used in clinical laboratory.

### SUMMARY OF THE INVENTION

The invention aims to provide an improved high-resolution HLA typing method that unambiguously resolves HLA class I (HLA-A, -B, -C) and class II (HLA-DRB1, -DRB3, -DRB4, -DRB5, -DQA1, -DQB1 and -DPB1) alleles at a level of resolution up to 8 digits and that is sufficiently simple and cost efficient to be routinely used in clinical laboratories.

Accordingly, in a first aspect, the present invention relates to a method for determining the HLA genotype of a DNA sample, comprising

a) contacting, in a reaction vessel, the DNA sample with an amplification reaction mixture comprising a set of amplification primers comprising, or consisting of, the entire sequences set forth in SEQ ID NOs: 1 to 14, or truncated forms thereof in which one to five, 1, 2, 3, 4 or 5, nucleotides are missing at their 5′ termini;

b) amplifying the targeted sequences using a primer-dependent DNA amplification reaction thereby producing amplicons; and

c) determining the sequence of said amplicons.

Step a) of the method may further comprise
contacting, in a distinct reaction vessel, the DNA sample with an amplification reaction mixture comprising a set of amplification primers comprising, or consisting of, the sequences set forth in SEQ ID NOs: 19 to 24 or truncated forms thereof in which one to five nucleotides are missing at their 5′ termini, targeting HLA-DRB1, HLA-DRB3, HLA-DRB4 and HLA-DRB5 genes; and/or
contacting, in a distinct reaction vessel, the DNA sample with an amplification reaction mixture comprising a set of amplification primers comprising, or consisting of, the sequences set forth in SEQ ID NOs: 25 to 28 or truncated forms thereof in which one to five nucleotides are missing at their 5′ termini, targeting HLA-DPB1 gene; and/or
contacting, in a distinct reaction vessel, the DNA sample with an amplification reaction mixture comprising a set of amplification primers comprising, or consisting of, the sequences set forth in SEQ ID NOs: 29 to 32 or truncated forms thereof in which one to five nucleotides are missing at their 5′ termini, targeting HLA-DQB1 gene; and/or
contacting, in a distinct reaction vessel, the DNA sample with an amplification reaction mixture comprising a set of amplification primers comprising, or consisting of, the sequences set forth in SEQ ID NOs: 33 to 36 or truncated forms thereof in which one to five nucleotides are missing at their 5′ termini, targeting HLA-DQA1 gene; and/or
contacting, in a distinct reaction vessel, the DNA sample with an amplification reaction mixture comprising a set of amplification primers comprising the sequences set forth in SEQ ID NOs: 13 and 14 or truncated forms thereof in which one to five nucleotides are missing at their 5′ termini, targeting HLA-A gene; and/or
contacting, in a distinct reaction vessel, the DNA sample with an amplification reaction mixture comprising a set of amplification primers comprising the sequences set forth in SEQ ID NOs: 15 to 17 or truncated forms thereof in which one to five nucleotides are missing at their 5′ termini, targeting HLA-B gene; and/or
contacting, in a distinct reaction vessel, the DNA sample with an amplification reaction mixture comprising a set of amplification primers comprising the sequences set forth in SEQ ID NOs: 15 and 18 or truncated forms thereof in which one to five nucleotides are missing at their 5′ termini, targeting HLA-C gene.

In step b) of the method, targeted sequences in each reaction vessel may be amplified using a uniform thermocycling profile. In particular, the annealing temperature may be in the range of 55 to 65° C.

Preferably, the primer-dependent DNA amplification reaction is a PCR reaction.

In step c) of the method, the sequences of amplicons may be determined using a next generation sequencing method.

The method may further comprise comparing the determined sequences of the amplicons with the DNA sequences of known HLA types.

In another aspect, the present invention also relates to a kit for determining the HLA genotype of a DNA sample, comprising a set of amplification primers comprising, or consisting of, the entire sequences set forth in SEQ ID NOs: 1 to 14 or truncated forms thereof in which one to five nucleotides are missing at their 5′ termini.

The kit may further comprise
a set of amplification primers comprising, or consisting of, the entire sequences set forth in SEQ ID NOs: 19 to 24 or truncated forms thereof in which one to five nucleotides are missing at their 5′ termini; and/or
a set of amplification primers comprising, or consisting of, the entire sequences set forth in SEQ ID NOs: 25 to 28 or truncated forms thereof in which one to five nucleotides are missing at their 5′ termini; and/or
a set of amplification primers comprising, or consisting of, the entire sequences set forth in SEQ ID NOs: 29 to 32 or truncated forms thereof in which one to five nucleotides are missing at their 5′ termini; and/or
a set of amplification primers comprising, or consisting of, the entire sequences set forth in SEQ ID NOs: 33 to 36 or truncated forms thereof in which one to five nucleotides are missing at their 5′ termini; and/or
a set of amplification primers comprising, or consisting of, the entire sequences set forth in SEQ ID NOs: 15 to 18 or truncated forms thereof in which one to five nucleotides are missing at their 5′ termini.

### BRIEF DESCRIPTION OF THE DRAWINGS

FIG. 1. Electrophoresis gels of PCR products obtained from PCR reactions with a mix of (i) CL1 F7 and CL1 R7 primers (HLA-A locus); (ii) CL1 F8, CL1 R8 and CL1 R9 primers (HLA-B locus); (iii) CL1 F8 and CL1 R10 primers (HLA-C locus); (iv) CL2 F1, CL2 F2, CL2 R1 to CL2 R3 and CL2 R12 primers (HLA-DRB1, -DRB3, -DRB4, -DRB5 loci); (v) CL2 F8, CL2 F9, CL2 R8 and CL2 R9 primers (HLA-DQA1 locus); (vi) CL2 F6, CL2 F7, CL2 R6 and CL2 R7 primers (HLA-DQB1 locus); (vii) CL2 F4, CL2 F5, CL2 R4 and CL2 R5 primers (HLA-DPB1 locus); or (viii) CL1 F1 to CL1 F7 and CL1 R1 to CL1 R7 primers (HLA-A, B and C loci).

FIG. 2. Results of HLA-A, -B, -C, -DRB1, -DQA1, -DQB1 and -DPB1 typing for 88 samples using the method of the invention. PCR amplification reactions were performed in the same device using the same cycling profile. Only five PCR reaction vessels were needed to assign HLA-A, -B, -C, -DRB1, -DQA1, -DQB1 and -DPB1 alleles at a level of resolution up to 8-digits (see also table 2) without any ambiguity.

FIG. 3. Electrophoresis gels of PCR products obtained from PCR reactions with a mix of the primers of SEQ ID NO: 37 and SEQ ID NO: 38 (HLA-DQB1 locus).

### DETAILED DESCRIPTION OF THE INVENTION

Based on their solid knowledge of HLA system polymorphism and clinical practical constraints, the inventors developed a new HLA typing method that is simpler, faster and more cost effective than currently known NGS-based methods.

The inventors identified different sets of amplification primers that dramatically reduces the number of amplification reactions needed to obtain appropriated amplicons for an extensive HLA typing which includes at least A, B and C loci, and optionally DRB1, DRB3, DRB4, DRB5, DQA1, DQB1 and DPB1 loci, with the highest level of resolution and without ambiguity.

The amplification primers were chosen to cover the entire gene from the 5′ untranslated region (UTR) to the 3′ UTR for loci A, B, C, DQA1 and DQB1 genes and from intron 1 to the 3′ UTR for DRB1, DRB3, DRB4, DRB5 and DPB1 genes.

The amplification reactions can be performed with a high level of multiplexing and a uniform thermocycling profile thereby decreasing the number of reaction vessels as well as thermocyclers needed.

As illustrated in the experimental section, the method of the invention completely resolves HLA class I (HLA-A, -B, -C) and class II (HLA-DRB1, -DRB3, -DRB4, -DRB5, -DQA1, -DQB1 and -DPB1) alleles at a level of resolution up to 8 digits without any ambiguity and allows detection of new HLA alleles as well as null alleles. These results demonstrate that this method overcomes current limitations in performing high-throughput and high-resolution HLA typing in clinical laboratories.

### DEFINITIONS

The term “allele” as used herein, refers to one of the alternative forms of a genetic locus. As used herein, the term “locus” refers to the position on a chromosome of a particular gene or allele.

The term “genotype” as used herein, refers to a description of the alleles of a gene or a plurality of genes contained in an individual or in a sample from said individual.

The expression “determining the HLA genotype” as used herein refers to determining the HLA polymorphisms present in the individual alleles of a subject.

The term “DNA sample” refers to a sample containing human genomic DNA obtained from a subject.

As used herein, the term “subject” refers to a human, including adult, child and human at the prenatal stage.

The term “amplification primer” as used herein refers to an oligonucleotide that is capable of selectively hybridizing to a target nucleic acid or “template”, more particularly capable of annealing to a DNA region adjacent to a target sequence to be amplified, and provides a point of initiation for template-directed synthesis of a polynucleotide complementary to the template catalyzed by a polymerase enzyme such as a DNA polymerase (polymerase chain reaction amplification). The primer is preferably a single-stranded oligodeoxyribonucleotide. An amplification primer is typically 15 to 40 nucleotides in length, preferably 15 to 30 nucleotides in length. The amplification primer may comprise a region being complementary to the HLA sequence of interest and a region that is not complementary to the HLA sequence of interest. In this case, the region complementary to the HLA sequence of interest is at least 15 nucleotides in length. Primers are often obtained as synthesized molecules and can be designed with wide range of molecular modifications, in particular at their 5′- or 3′-terminus.

As used herein, the phrase “selectively hybridizing to” refers to the binding, duplexing, or hybridizing of an amplification primer only to a particular nucleotide sequence with a higher affinity, e.g., under more stringent conditions, than to other nucleotide sequences. One of skill in the art will appreciate that specific hybridization between nucleotides usually relies on Watson-Crick pair bonding between complementary nucleotide sequences.

The term “set of amplification primers” as used herein refers to at least two amplification primers, i.e. at least one forward primer and at least one reverse primer. The terms “forward primer” and “reverse primer” are used as understood in the art to refer to the set of primers used to amplify both strands of a double-stranded nucleic acid.

As used herein, the term “5′ truncated form” refers to a primer comprising at least 15 nucleotides and wherein, by comparison to the reference sequence, e.g. one of the sequences set forth in SEQ ID NOs: 1 to 36, one or several nucleotides are missing at the 5′ terminus. In a particular embodiment, the term “5′ truncated form” refers to a primer comprising at least 15 nucleotides and wherein, by comparison to the reference sequence, 1, 2, 3, 4 or 5 nucleotides are missing at the 5′ terminus, preferably 1, 2 or 3 nucleotides, more preferably 1 or 2 nucleotides.

The term “primer-dependent DNA amplification reaction”, as used herein, refers to an enzymatic process of growth of nucleic acid molecules that needs polymerase enzyme, template molecule annealed with amplification primers as well as nucleotides and adequate environmental conditions. Examples of amplification techniques include, but are not limited to, polymerase chain reaction (PCR), modified PCR techniques and ligase chain reaction (LCR). Typically, the segment is defined by a forward primer and a reverse primer that hybridize to the 5′ end and 3′ end of the segment to be amplified. Conditions and reagents for primer extension reactions are well known in the art (see for example Sambrook et al. Molecular Cloning, A Laboratory Manual, Third Edition, Cold Spring Harbor Laboratory Press, 2000, and Ausubel et al. In Current Protocols in Molecular Biology, John Wiley & Sons, NY, 1998). Amplification reaction can comprise thermal-cycling or can be performed isothermally. Preferably the primer-dependent DNA amplification reaction is a polymerase chain reaction (PCR). Preferably, PCR is performed in a thermocycler.

The term “polymerase chain reaction” or “PCR” as used herein refers to a method for amplifying a DNA sequence using a heat-stable DNA polymerase and a set of amplification primers in a cyclical reaction where the annealing of primers, synthesis of progeny strand DNA and denaturation of the duplexes, are each conducted at different temperatures. Because the newly synthesized DNA strands can subsequently serve as additional templates for the same primer sequences, successive rounds of primer annealing, strand elongation and dissociation produce rapid amplification of the target sequence.

As used herein, the term “amplification reaction mixture” refers to a mixture comprising all reagents needed for performing primer-dependent DNA amplification reaction. Typically, this mixture comprises a DNA polymerase, a set of amplification primers, an appropriate buffer and dNTPs.

As used herein, the term “DNA polymerase” refers to an enzyme that is essential for elongation of amplification primers in nucleic acid templates. The skilled person may easily choose a convenient polymerase enzyme based on its characteristics such as efficiency, processivity or fidelity. Preferably, the polymerase is a high-fidelity and heat-stable polymerase.

The term “amplicon” or “amplification product” as used herein refers to a fragment of DNA spanned within a pair of amplification primers, this fragment being amplified exponentially by a DNA polymerase. An amplicon can be single-stranded or double-stranded.

The expression “determining the sequence” as used herein, refers to the process of determining the identity of nucleotide bases at each position along the length of a polynucleotide. Any sequencing method can be used in the present invention.

As used herein, the terms “nucleic acid molecule”, “oligonucleotide” and “polynucleotide” are used interchangeably and refer to single-stranded or double-stranded polymers of nucleotide monomers, preferably DNA, linked by phosphodiester bonds.

As used in this specification, the term “about” refers to a range of values ±10% of the specified value. For example, “about 20” includes ±10% of 20, and refers to from 18 to 22. Preferably, the term “about” refers to a range of values ±5% of the specified value.

Preferably, the methods of the invention are in vitro methods.

HLA Genes

HLA-A, HLA-B and HLA-C are the three major types of human MHC class I cell surface antigen-presenting proteins. They play a central role in the immune system by presenting peptides derived from the endoplasmic reticulum lumen and are expressed in nearly all cells. These receptors are heterodimers and are composed of a heavy a chain and a light chain (an invariant 132 microglobulin molecule coded for by a separate region of the human genome). The HLA-A gene (Gene ID: 3105) contain 8 coding exons, the HLA-B gene (Gene ID: 3106) and the HLA-C gene (Gene ID: 3107) contain 7 coding exons.

HLA class II molecules are heterodimers consisting of an alpha chain and a beta chain, both anchored in the membrane. They play a central role in the immune system by presenting peptides derived from extracellular proteins. Class II molecules are expressed in antigen presenting cells (e.g. B lymphocytes, dendritic cells, macrophages).

HLA-DRB1 (Gene ID: 3123), HLA-DRB3 (Gene ID: 3125), HLA-DRB4 (Gene ID: 3126) and HLA-DRB5 (Gene ID: 3127) belong to the HLA class II beta chain paralogs. The heterodimers consist of an alpha chain (DRA) and a beta chain (DRB). The beta chain is approximately 26-28 kDa and is encoded by 6 exons.

HLA-DQA1 (Gene ID: 3117) belongs to the HLA class II alpha chain paralogues. The heterodimers consist of an alpha chain (DQA) and a beta chain (DQB). The alpha chain is approximately 33-35 kDa and is encoded by 4 coding exons.

HLA-DQB1 (Gene ID: 3119) belongs to the HLA class II beta chain paralogs. The beta chain is approximately 26-28 kDa and is encoded by 5 coding exons.

HLA-DPB1 (Gene ID: 3115) belongs to the HLA class II beta chain paralogues. The heterodimers consist of an alpha chain (DPA) and a beta chain (DPB). The beta chain is approximately 26-28 kDa and is encoded by 5 coding exons.

In a first aspect, the present invention thus relates to a method for determining the HLA genotype of a DNA sample, comprising

a) contacting, in a reaction vessel, the DNA sample with an amplification reaction mixture comprising a set of amplification primers comprising, or consisting of, the entire sequences set forth in SEQ ID NOs: 1 to 14, or 5′ truncated forms thereof;

b) amplifying the targeted sequences using a primer-dependent DNA amplification reaction thereby producing amplicons; and

c) determining the sequence of said amplicons.

The DNA sample used in the method of the invention comprises, or consists of, human genomic DNA that can be obtained from any suitable source. Typically, genomic DNA is obtained from blood sample or a buccal swab sample. Preferably, genomic DNA sample is extracted from peripheral blood mononuclear cells. A large number of methods are available and well-known by the skilled person for the isolation and purification of genomic DNA samples. Any method suitable to provide DNA sample that can be used in amplification reaction such as PCR or sequencing reaction, can be used in the present invention. Preferably, the DNA sample should be free of any protein or other contaminants that could inhibit amplification or sequencing reactions.

In step a), the DNA sample is contacted in a reaction vessel with an amplification reaction mixture comprising a set of amplification primers. The amount of genomic DNA in the reaction can vary between 5 to 500 ng DNA per 50 μL reaction. This amount can be easily adjusted by the skilled person. Preferably, about 160 ng genomic DNA were used per 50 μL reaction or 80 ng genomic DNA per 20 μL reaction.

In the method of the invention, the set of amplification primers targeting HLA-A, HLA-B and HLA-C genes comprises
at least the following forward primers: a primer comprising, or consisting of, the entire sequence set forth in SEQ ID NO: 1, or a 5′ truncated form thereof; a primer comprising, or consisting of, the entire sequence set forth in SEQ ID NO: 2, or a 5′ truncated form thereof; a primer comprising, or consisting of, the entire sequence set forth in SEQ ID NO: 3, or a 5′ truncated form thereof; a primer comprising, or consisting of, the entire sequence set forth in SEQ ID NO: 4, or a 5′ truncated form thereof; a primer comprising, or consisting of, the entire sequence set forth in SEQ ID NO: 5, or a 5′ truncated form thereof; a primer comprising, or consisting of, the entire sequence set forth in SEQ ID NO: 6, or a 5′ truncated form thereof; and a primer comprising, or consisting of, the entire sequence set forth in SEQ ID NO: 13, or a 5′ truncated form thereof; and
at least the following reverse primers: a primer comprising, or consisting of, the entire sequence set forth in SEQ ID NO: 7, or a 5′ truncated form thereof; a primer comprising, or consisting of, the entire sequence set forth in SEQ ID NO: 8, or a 5′ truncated form thereof; a primer comprising, or consisting of, the entire sequence set forth in SEQ ID NO: 9, or a 5′ truncated form thereof; a primer comprising, or consisting of, the entire sequence set forth in SEQ ID NO: 10, or a 5′ truncated form thereof; a primer comprising, or consisting of, the entire sequence set forth in SEQ ID NO: 11, or a 5′ truncated form thereof; a primer comprising, or consisting of, the entire sequence set forth in SEQ ID NO: 12, or a 5′ truncated form thereof; and a primer comprising, or consisting of, the entire sequence set forth in SEQ ID NO: 14, or a 5′ truncated form thereof.

In a particular embodiment, the set of amplification primers targeting HLA-A, HLA-B and HLA-C genes comprises primers comprising, or consisting of, the sequences set forth in SEQ ID NOs: 1 to 14.

In a preferred embodiment, the set of amplification primers comprises primers consisting of the sequences set forth in SEQ ID NOs: 1 to 14, i.e. the forward primers SEQ ID NOs: 1, 2, 3, 4, 5, 6 and 13 and the reverse primers SEQ ID NOs: 7, 8, 9, 10, 11, 12 and 14.

The method of the invention may also be used to determine the genotype of HLA-DRB1, -DRB3, -DRB4, -DRB5 genes and/or HLA-DPB1 gene and/or HLA-DQB1 and/or HLA-DQA1 and/or to separately determine the genotype of HLA-A, HLA-B and/or HLA-C.

Thus, in an embodiment, the method may further comprise in step a),
contacting, in a distinct reaction vessel, the DNA sample with an amplification reaction mixture comprising a set of amplification primers comprising, or consisting of, the sequences set forth in SEQ ID NOs: 19 to 24, or 5′ truncated forms thereof, targeting HLA-DRB1, HLA-DRB3, HLA-DRB4 and HLA-DRB5 genes, i.e. a primer comprising, or consisting of, the entire sequence set forth in SEQ ID NO: 19, or a 5′ truncated form thereof; a primer comprising, or consisting of, the entire sequence set forth in SEQ ID NO: 20, or a 5′ truncated form thereof; a primer comprising, or consisting of, the entire sequence set forth in SEQ ID NO: 21, or a 5′ truncated form thereof; a primer comprising, or consisting of, the entire sequence set forth in SEQ ID NO: 21, or a 5′ truncated form thereof; a primer comprising, or consisting of, the entire sequence set forth in SEQ ID NO: 22, or a 5′ truncated form thereof; a primer comprising, or consisting of, the entire sequence set forth in SEQ ID NO: 23, or a 5′ truncated form thereof; and a primer comprising, or consisting of, the entire sequence set forth in SEQ ID NO: 24, or a 5′ truncated form thereof; and/or
contacting, in a distinct reaction vessel, the DNA sample with an amplification reaction mixture comprising a set of amplification primers comprising, or consisting of, the sequences set forth in SEQ ID NOs: 25 to 28, or 5′ truncated forms thereof, targeting HLA-DPB1 gene, i.e. a primer comprising, or consisting of, the entire sequence set forth in SEQ ID NO: 25, or a 5′ truncated form thereof; a primer comprising, or consisting of, the entire sequence set forth in SEQ ID NO: 26, or a 5′ truncated form thereof; a primer comprising, or consisting of, the entire sequence set forth in SEQ ID NO: 27, or a 5′ truncated form thereof; and a primer comprising, or consisting of, the entire sequence set forth in SEQ ID NO: 28, or a 5′ truncated form thereof; and/or
contacting, in a distinct reaction vessel, the DNA sample with an amplification reaction mixture comprising a set of amplification primers comprising, or consisting of, the sequences set forth in SEQ ID NOs: 29 to 32, or 5′ truncated forms thereof, targeting HLA-DQB1 gene, i.e. a primer comprising, or consisting of, the entire sequence set forth in SEQ ID NO: 29, or a 5′ truncated form thereof; a primer comprising, or consisting of, the entire sequence set forth in SEQ ID NO: 30, or a 5′ truncated form thereof; a primer comprising, or consisting of, the entire sequence set forth in SEQ ID NO: 31, or a 5′ truncated form thereof; and a primer comprising, or consisting of, the entire sequence set forth in SEQ ID NO: 32, or a 5′ truncated form thereof; and/or
contacting, in a distinct reaction vessel, the DNA sample with an amplification reaction mixture comprising a set of amplification primers comprising, or consisting of, the sequences set forth in SEQ ID NOs: 33 to 36, or 5′ truncated forms thereof, targeting HLA-DQA1 gene, i.e. a primer comprising, or consisting of, the entire sequence set forth in SEQ ID NO: 33, or a 5′ truncated form thereof; a primer comprising, or consisting of, the entire sequence set forth in SEQ ID NO: 34, or a 5′ truncated form thereof; a primer comprising, or consisting of, the entire sequence set forth in SEQ ID NO: 35, or a 5′ truncated form thereof; and a primer comprising, or consisting of, the entire sequence set forth in SEQ ID NO: 36, or a 5′ truncated form thereof; and/or
contacting, in a distinct reaction vessel, the DNA sample with an amplification reaction mixture comprising a set of amplification primers comprising the sequences set forth in SEQ ID NOs: 13 and 14 or 5′ truncated forms thereof, targeting HLA-A gene, i.e. a primer comprising, or consisting of, the entire sequence set forth in SEQ ID NO: 13, or a 5′ truncated form thereof; and a primer comprising, or consisting of, the entire sequence set forth in SEQ ID NO: 14, or a 5′ truncated form thereof; and/or
contacting, in a distinct reaction vessel, the DNA sample with an amplification reaction mixture comprising a set of amplification primers comprising the sequences set forth in SEQ ID NOs: 15 to 17 or 5′ truncated forms thereof, targeting HLA-B gene, i.e. a primer comprising, or consisting of, the entire sequence set forth in SEQ ID NO: 15, or a 5′ truncated form thereof; a primer comprising, or consisting of, the entire sequence set forth in SEQ ID NO: 16, or a 5′ truncated form thereof; and a primer comprising, or consisting of, the entire sequence set forth in SEQ ID NO: 17, or a 5′ truncated form thereof; and/or
contacting, in a distinct reaction vessel, the DNA sample with an amplification reaction mixture comprising a set of amplification primers comprising the sequences set forth in SEQ ID NOs: 15 and 18 or 5′ truncated forms thereof, targeting HLA-C gene, i.e. a primer comprising, or consisting of, the entire sequence set forth in SEQ ID NO: 15, or a 5′ truncated form thereof; and a primer comprising, or consisting of, the entire sequence set forth in SEQ ID NO: 18, or a 5′ truncated form thereof.

In this embodiment, each set of amplification primers may comprise
primers comprising, or consisting of, the sequences set forth in SEQ ID NOs: 19 to 24; or
primers comprising, or consisting of, the sequences set forth in SEQ ID NOs: 25 to 28; or
primers comprising, or consisting of, the sequences set forth in SEQ ID NOs: 29 to 32; or
primers comprising, or consisting of, the sequences set forth in SEQ ID NOs: 33 to 36; or
primers comprising, or consisting of, the sequences set forth in SEQ ID NOs:13 and 14; or
primers comprising, or consisting of, the sequences set forth in SEQ ID NOs: 15 to 17; or
primers comprising, or consisting of, the sequences set forth in SEQ ID NOs: 15 and 18.

Alternatively, in this embodiment, each set of amplification primers may comprise
primers consisting of the sequences set forth in SEQ ID NOs: 19 to 24; or
primers consisting of the sequences set forth in SEQ ID NOs: 25 to 28; or
primers consisting of the sequences set forth in SEQ ID NOs: 29 to 32; or
primers consisting of the sequences set forth in SEQ ID NOs: 33 to 36; or
primers consisting of the sequences set forth in SEQ ID NOs:13 and 14; or
primers consisting of the sequences set forth in SEQ ID NOs: 15 to 17; or
primers consisting of the sequences set forth in SEQ ID NOs: 15 and 18.

An amplification primer as used in the present invention may comprise, or consist of, one of the entire sequences set forth in SEQ ID NOs: 1 to 36, and one or several additional nucleotides at the 5′ end and/or 3′ end, preferably from 1 to 10 additional nucleotides, more preferably 1, 2, 3, 4 or 5 additional nucleotides. An amplification primer may also comprise, or consist of, one of the 5′ truncated form of one of the sequences set forth in SEQ ID NOs: 1 to 36, and one or several additional nucleotides at the 5′ end and/or 3′ end. In particular, the primer may comprise additional nucleotides at the 3′ end that are complementary to the HLA sequence of interest. The primer may comprise additional nucleotides at the 5′ end that are complementary or not to the HLA sequence of interest. In particular, the primer may comprise 1 to 10, i.e. 1, 2, 3, 4, 5, 6, 7, 8, 9 or 10, additional nucleotides, preferably 1, 2, 3, 4 or 5 additional nucleotides at the 5′ end of one of the sequences set forth in SEQ ID NOs: 1 to 36. Additional nucleotides at the 5′ end may include for example a restriction site or an identification tag. An identification tag that is sequenced with the amplicon, can be used to mark the HLA amplicons from each individual (or sample) who is being tested. An identification tag is usually from 4 to 10 nucleotides in length, preferably from 4 to 5 nucleotides in length.

In a particular embodiment, a primer comprising one of the entire sequences set forth in SEQ ID NOs: 1 to 36 or a 5′ truncated form thereof, comprises (i) one to five additional nucleotides at the 5′ end and/or one to five additional nucleotides at the 3′ end, or (ii) one to ten additional nucleotides at the 5′ end.

In a more particular embodiment, an amplification primer as used in the present invention may comprise, or consist of, one of the entire sequences set forth in SEQ ID NOs: 1 to 36, and one or several additional nucleotides at the 5′ end, preferably from 1 to 10 additional nucleotides, more preferably 1, 2, 3, 4 or 5 additional nucleotides. For example, HLA-DQB1 gene may be amplified using a set of amplification primers wherein the primer of SEQ ID NO: 30 is replaced with the primer of SEQ ID NO: 37, i.e. the primer of SEQ ID NO: 30 with four additional nucleotides at the 5′ end, and/or wherein the primer of SEQ ID NO: 31 is replaced with the primer of SEQ ID NO: 38, i.e. the primer of SEQ ID NO: 31 with six additional nucleotides at the 5′ end. Thus, optionally, in the methods of the invention, the primer of SEQ ID NO: 30 may be replaced with the primer of SEQ ID NO: 37, and/or the primer of SEQ ID NO: 31 may be replaced with the primer of SEQ ID NO: 38.

When increasing or decreasing the length of a primer, parameters such as G/C content, prevention of internal secondary structure and primer dimers as well as melting temperature (Tm) should be considered.

The reaction vessel may be any suitable vessel, preferably a PCR tube (e.g. 0.2 mL or 0.5 mL) that can be used in a thermocycler.

The content of the amplification reaction mixture may be adapted according to the type of amplification reaction used in step b).

In a particular embodiment, the mixture comprises a heat-stable DNA polymerase and an appropriate buffer (typically provided with the DNA polymerase), a set of amplification primers and dNTPs. Preferably the DNA polymerase is a high-fidelity DNA polymerase, i.e. with an error rate less than 10⁻⁵, more preferably less than 10⁻⁶. Examples of suitable available DNA polymerases include, but are not limited to, Pyrococcus furiosus (Pfu) DNA polymerase (Stratagene), Phusion™ DNA Polymerase (New England Biolabs), Platinum® Taq DNA Polymerase High Fidelity (Life Technologies), PfuUltra™ (Stratagene), or MyFi™ DNA polymerase (Bioline).

In step b) of the method of the invention, HLA targeted sequences are amplified using a primer-dependent DNA amplification reaction thereby producing amplicons. The sets of amplification primers identified by the inventors allows the amplification of (i) HLA-A, -B, -C, (ii) -DRB1, -DRB3, -DRB4, -DRB5, (iii) -DPB1, (iv) -DQB1 and (v) -DQA1 in only five distinct reaction vessels using a uniform thermocycling profile.

The HLA amplicons may be obtained using any type of amplification reaction. Preferably the primer-dependent DNA amplification reaction is a polymerase chain reaction (PCR) and is preferably performed in a thermocycler. Preferably, all primer-dependent DNA amplification reactions are performed in the same thermocycler. However, each amplification reaction can also be performed independently.

The thermocycling profile comprises an initial denaturation step to fully melt the template, i.e. the genomic DNA contained in the sample. This initial denaturation lasts at least 1 minute at 95° C., preferably up to 3 min.

After this initial denaturation, 25 to 40 cycles are performed, each cycle consisting of DNA denaturation, annealing reaction and elongation/extension reaction.

DNA denaturation is usually performed at 94° C. to 96° C. for 15 to 30 sec.

The optimal annealing temperature depends on the set of amplification primers. The primers used in the method of the invention were designed to efficiently anneal to the target sequences at the same temperature. Preferably, the annealing temperature is in the range of 55 to 65° C., more preferably in the range of 58 to 62° C., even more preferably is 60° C.

In a particular embodiment, the amplification primers are selected from primers consisting of the entire sequences set forth in SEQ ID NOs: 1 to 36, and the annealing temperature is in the range of 55 to 65° C., preferably is in the range of 58 to 62° C., more preferably is 60° C.

The extension temperature depends on the DNA polymerase used. Usually, this temperature is about 72° C. However, some DNA polymerases may require adjustments. The extension time depends on the length of the amplicon and the speed of the polymerase and can be easily determined by the skilled person. Preferably, the elongation reaction is performed at about 72° C. for 1 to 5 minutes.

Optionally, amplification products can be purified and/or quantified before sequencing. If necessary, the concentration can be adjusted.

In step c) of the method of the invention, the sequences of amplicons obtained in step b) are determined.

Any known sequencing method can be used to determine the sequences of amplicons, such as the Sanger method or a next-generation sequencing (NGS) method. As used herein, the term “NGS method” refers to any high-throughput sequencing technology that parallelizes the sequencing process, producing thousands or millions of sequences concurrently. Preferably, the sequences are determined using a NGS method. Examples of NGS methods include, but are not limited to, pyrosequencing (Roche Diagnostics), Illumina (Solexa, MIseq, NextSeq 500) sequencing or SOLiD sequencing (Applied Biosystems) Ion torrent (Life Technology). All these methods are well known by the skilled person and can be easily performed according to the manufacturer instructions.

The sequences can be analyzed using suitable software, preferably a software that is able to filter out related sequence reads (such as other unwanted HLA genes) that could be co-amplified with the target sequences. The software can be used to merge sequences together, to compare to HLA sequences database and to propose a genotype for each loci.

Once the relevant DNA sequences have been obtained, the assignment of genotypes at each locus based on the available sequences is performed by comparing said sequences with the DNA sequences of known HLA types, preferably with an HLA sequence database (e.g the IMGT/HLA Database or the dbMHC database) using a suitable software (e.g MPS or Omixon). Null alleles as well as new alleles can also be detected.

In a further aspect, the present invention relates to a kit for determining the HLA genotype of a DNA sample, comprising a set of amplification primers comprising, or consisting of, the entire sequences set forth in SEQ ID NOs: 1 to 14 or 5′ truncated forms thereof.

In a preferred embodiment, the kit comprises a set of amplification primers consisting of the sequences set forth in SEQ ID NOs: 1 to 14.

In a particular embodiment, the kit further comprises
a set of amplification primers comprising, or consisting of, the entire sequences set forth in SEQ ID NOs: 19 to 24 or 5′ truncated forms thereof; and/or
a set of amplification primers comprising, or consisting of, the entire sequences set forth in SEQ ID NOs: 25 to 28 or 5′ truncated forms thereof; and/or
a set of amplification primers comprising, or consisting of, the entire sequences set forth in SEQ ID NOs: 29 to 32 or 5′ truncated forms thereof; and/or
a set of amplification primers comprising, or consisting of, the entire sequences set forth in SEQ ID NOs: 33 to 36 or 5′ truncated forms thereof; and/or
a set of amplification primers comprising, or consisting of, the entire sequences set forth in SEQ ID NOs: 15 to 18 or 5′ truncated forms thereof.

In another particular embodiment, the kit further comprises
a set of amplification primers consisting of the sequences set forth in SEQ ID NOs: 19 to 24; and/or
a set of amplification primers consisting of the sequences set forth in SEQ ID NOs: 25 to 28; and/or
a set of amplification primers consisting of the sequences set forth in SEQ ID NOs: 29 to 32; and/or
a set of amplification primers consisting of the sequences set forth in SEQ ID NOs: 33 to 36; and/or
a set of amplification primers consisting of the sequences set forth in SEQ ID NOs: 15 to 18.

Optionally, in the kits of the invention, the primer of SEQ ID NO: 30 may be replaced with the primer of SEQ ID NO: 37, and/or the primer of SEQ ID NO: 31 may be replaced with the primer of SEQ ID NO: 38.

All variations in the primer sequences and lengths described above for the method of the invention are also contemplated in this aspect.

Optionally, the kit may also comprise a DNA polymerase and/or dNTPs and/or buffers and/or sequencing reagents and/or reagents needed to extract genomic DNA and/or a leaflet providing guidelines to use such a kit.

The present invention also relates to the use of a kit according to the invention for determining the HLA genotype of a DNA sample.

Further aspects and advantages of the present invention will be described in the following examples, which should be regarded as illustrative and not limiting.

### Examples

Human genomic DNA samples were obtained from peripheral blood cells according to standard methods, for example using a commercial kit (e.g. DNA whole blood kit, Kurabo) and following the manufacturer's instructions.

A set of amplification primers was chosen to cover the entire gene from the 5′ UT to the 3′ UT region for loci A, B, C, DQA1 and DQB1 genes and from intron 1 to the 3′ UT region for DRB1, DRB3, DRB4, DRB5 and DPB1 genes.

Sequences and positions on the human genome of the primers are described in table 1 below. The respective position of each primer is determined by the length between the 3′ extremity of the primer and the extremity of the exon indicated in the table 1. CLx_Fx primers are forward primers and CLx Rx primers are reverse primers.

TABLE 1
Amplification primers

| | | | SEQ |
|---|---|---|---|
| | | | ID |
| Name | Sequence (5′->3′) | Position | NOs |
| CL1_ | CGGGGCTCTCAGGGTCTCAGG | 228 b 5′ of exon | 1 |
| F1 | CTCC | 1_HLA*C07:01 | |
| CL1_ | CGTGGCTCTCAGGGTCTCAGG | 235 b 5′ of exon | 2 |
| F2 | CCCC | 1_HLA*A01:01 | |
| CL1_ | TTCCCACTCCCATTGGGTGTC | 87 b 5′ of exon | 3 |
| F3 | GGGT | 1_HLA*C07:01 | |
| CL1_ | TTCCCACTCCCATTGGGTGTC | 88 b 5′ of exon | 4 |
| F4 | GGAT | 1_HLA*B08:01 | |
| CL1_ | TTCCCACTCCCATTGGGTATT | 88 b 5′ of exon | 5 |
| F5 | GGAT | 1_HLA*B07:02 | |
| CL1_ | TTCTCACTCCCATTGGGTGTC | 89 b 5′ of exon | 6 |
| F6 | GGGT | 1_HLA*A01:01 | |
| CL1_ | GCCTTTGCAGAAAGAGATGCC | 252 b 3′ of exon | 7 |
| R1 | AGAGGC | 7_HLA*B07:02 | |
| CL1_ | GTGCCTTTGCAGAAACAAAGT | 66 b 3′ of exon | 8 |
| R2 | CAGGGT | 8_HLA*A01:01 | |
| CL1_ | GCCTTTGTAGAAAGAGATGCC | 232 b 3′ of exon | 9 |
| R3 | AGAGGC | 7_HLA*C02:02 | |
| CL1_ | CAGTCCCACACAGGCAGCTGT | 169 b 3′ of exon | 10 |
| R4 | C | 7_HLA*C07:01 | |
| CL1_ | AGTCCCACACAAGGCAGCTGT | 1 b 3′ of exon | 11 |
| R5 | C | 8_HLA*A01:01 | |
| CL1_ | GGTCCCTCACAAGACAGCTGT | 177 b 3′ of exon | 12 |
| R6 | C | 7_HLA*B18:01 | |
| CL1_ | CTCCGCAGTTTCTTTTCTCCCT | 164 b 5′ of exon | 13 |
| F7 | CT | 1_HLA*A01:01 | |
| CL1_ | TTCAAGTCACAAAGGGAAGG | 40 b 3′ of exon | 14 |
| R7 | GCAG | 8_HLA*A01:01 | |
| CL1_ | CCCCACTCCCCTGAGTTTCAC | 169 b 5′ of exon | 15 |
| F8 | T | 1_HLA*B08:01 | |
| CL1_ | CATCTCAGTCCCTCACAAGA | 195 b 3′ of exon | 16 |
| R8 | | 7_HLA*B07:02 | |
| CL1_ | CATCTCGGTCCCTCACAAGA | 185 b 3′ of exon | 17 |
| R9 | | 7_HLA*B18:01 | |
| CL1_ | CATCTCAGTCCCACACAGGCA | 172 b 3′ of exon | 18 |
| R10 | GC | 7_HLA*C02:02 | |
| CL2_ | CCAGNRGASTGGAGAGGTCTG | 475 b 5′ of exon | 19 |
| F1 | TTTTCC | 2_DRB1*01:01 | |
| CL2_ | TCACTGCTCTTTAAGCTCCCCC | 499 b 5′ of exon | 20 |
| F2 | A | 2_DRB1*01:01 | |
| CL2_ | AGCCACAGGGGAGGACATTTT | 132 b 3′ of exon | 21 |
| R1 | CTGCA | 6_DRB1*07:01 | |
| CL2_ | AGCCACAAGGGAGGACATTTT | 130 b 3′ of exon | 22 |
| R2 | CTGCA | 6_DRB1*01:01 | |
| CL2_ | AGCCACAAGGATGGACATTTT | 132 b 3′ of exon | 23 |
| R3 | CTGCA | 6_DRB5*01:14 | |
| CL2_ | CTGAGGAAGCCACAAGGGAG | 139 b 3′ of exon | 24 |
| R12 | GACA | 6_DRB1*01:01 | |
| CL2_ | GATGAGAGTGGCGCCTCCGCT | 20 b 5′ of exon | 25 |
| F4 | CA | 2_DPB1*04:01 | |
| CL2_ | CAGCCCTGGGTGGGAAGATTT | 84 b 5′ of exon | 26 |
| F5 | GGGA | 2_DPB1*04:01 | |
| CL2_ | TTCCCTTCCTGGAGGAGCCTC | 76 b 3′ of exon | 27 |
| R4 | AGT | 5_DPB1*04:01 | |
| CL2_ | TGATCTCTGCTTCCTTCAGCA | 165 b 3′ of exon | 28 |
| R5 | ATGGA | 5_DPB1*04:01 | |
| CL2_ | CAGCTCCAGTGCTGATTGGTT | 128 b 5′ of exon | 29 |
| F6 | | 1_DQB1*02:01 | |
| CL2_ | CCAGSTACATCAGATCCATCA | 39 b 5′ of exon | 30 |
| F7 | GGTC | 1_DQB1*02:01 | |
| CL2_ | CGTGACAGCCACTGTAGGACT | 107 b 3′ of exon | 31 |
| R6 | | 6_DQB1*02:01 | |
| CL2_ | GGGGATGAAAGGAGATGACC | 136 b 3′ of exon | 32 |
| R7 | T | 6_DQB1*02:01 | |
| CL2_ | AAGGGGATTGCCCYGTCTCCT | 181 b 5′ of exon | 33 |
| F8 | TCCA | 1_DQA1*01:02 | |
| CL2_ | GGCAGGGTTTGGTTTGGGTGT | 76 b 5′ of exon | 34 |
| F9 | CTTCA | 1_DQA1*01:02 | |
| CL2_ | GCCACTTCCCAATTCCCCTAC | 104 b 3′ of exon | 35 |
| R8 | AACT | 4_DQA1*01:02 | |
| CL2_ | GCACCTGCAACAGGRCAGAC | 165 b 3′ of exon | 36 |
| R9 | ATGAGA | 4_DQA1*01:02 | |

All the classical Class I genes, i.e. HLA-A, B and C were amplified in the same PCR reaction using the following mix of primers: CL1 F1 to CL1 F7 and CL1 R1 to CL1 R7.

Considering HLA Class II loci, HLA-DRB1, DRB3, DRB4 and DRB5 loci were amplified in the same PCR reaction using the following set of primers: CL2 F1 and CL2 F2+CL2 R1 to R3 and CL2 R12.

Thus, a complete HLA-A, -B, -C, -DRB1, -DRB3, -DRB4, -DRB5 typing was obtained from only two PCR reactions performed in the same conditions.

Furthermore, HLA-DQA1, -DQB1 and -DPB1 loci were amplified using the following set of primers:
HLA-DQA1 locus was amplified using: CL2 F8 and CL2 F9+CL2 R8 and CL2 R9;
HLA-DQB1 locus was amplified using: CL2 F6 and CL2 F7+CL2 R6 and CL2 R7; and
HLA-DPB1 locus was amplified using: CL2 F4 and CL2 F5+CL2 R4 and CL2 R5.

A complete HLA-A, -B, -C, -DRB1, -DRB3, -DRB4, -DRB5, -DQA1, -DQB1 and -DPB1 typing was thus obtained from only five PCR reactions performed in the same conditions.

In addition, each Class I locus was separately and specifically amplified with the following set of primers:
locus HLA-A: CL1 F7 and CL1 R7,
locus HLA-B: CL1 F8 and CL1 R8+CL1 R9, and
locus HLA-C: CL1 F8 and CL1 R10.

The unique experimental PCR condition was as follow:

After a step at 95° C. for 3 min, 36 cycles of
denaturing at 96° C. for 20 sec
annealing at 60° C. for 30 sec
elongation at 72° C. For 5 min.

The PCR reaction was performed in a total volume of 20 μl, containing 0.1 μL of 25 μM each primer, 2 μl of dNTP (2 mM), 80 ng DNA, 2 U long range PCR enzyme (MyFi™ DNA Polymerase, Bioline) and 1× corresponding reaction buffer.

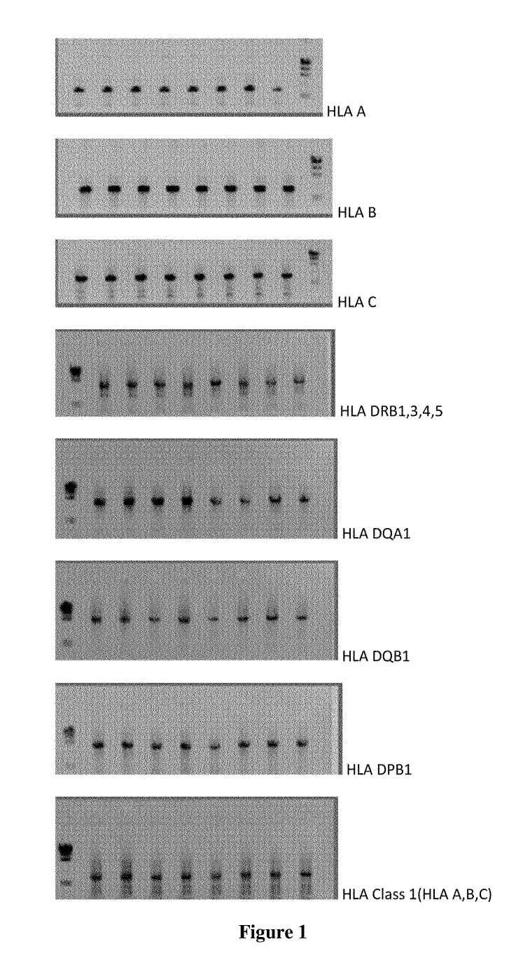

These PCR conditions were validated by a control of the PCR products on a gel. For illustration, an example of eight DNAs amplified with each mix of primers described above is presented on FIG. 1.

In order to confirm the specificity of the PCR products, the inventors sequenced these PCR products by using a so called-next generation sequencing method. For this purpose, among the different NGS technologies so far available, they have chosen to use Illumina System.

88 samples were analysed for HLA-A, -B, -C, -DRB1, -DQB1, -DQA1 and -DPB1 loci using the above-described set of primers. For the library preparation, the Nextera® XT DNA Kit from Illumina was used following instructions for use.

This step was followed by a tagging step using the Nextera® XT Index Kit before a final sequencing step using the MI Seq Reagent nano Kit V2 (300 cycles) from Illumina (Mi Seq System).

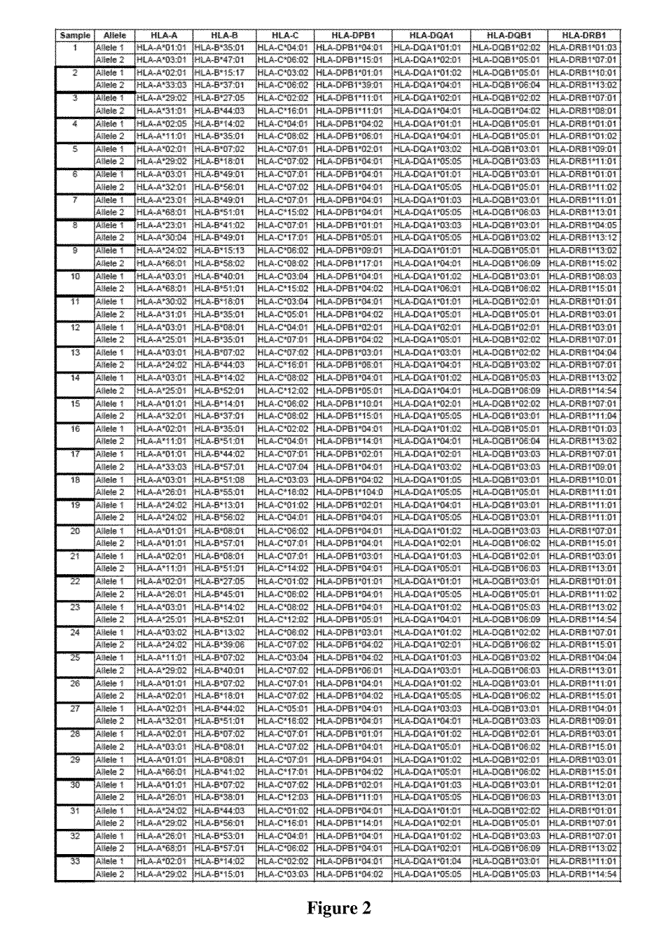

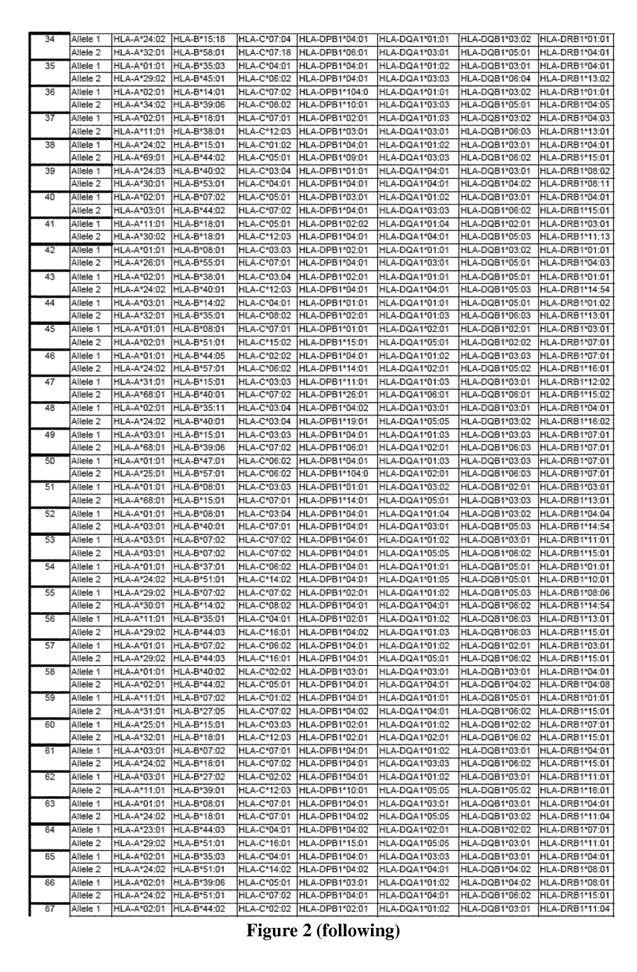

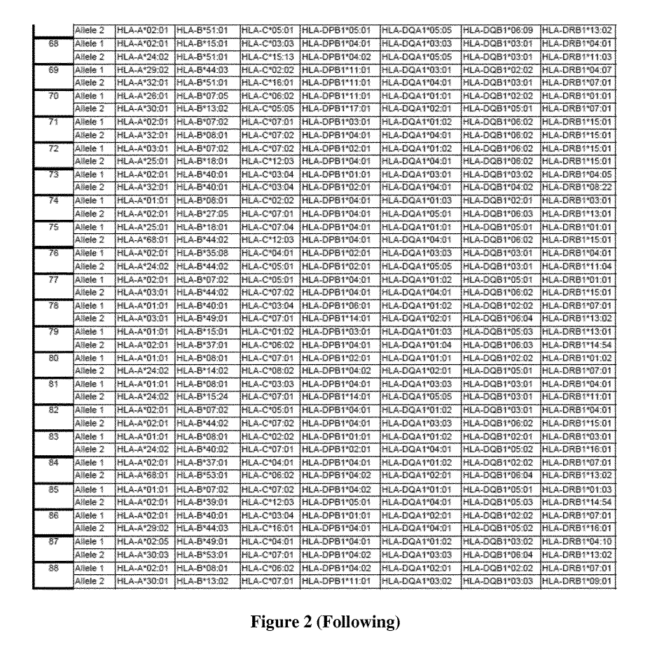

The sequences were analysed by the MPS V1 software from CONEXIO Genomics and OMIXON software. Genomic results were obtained for all 88 samples without any ambiguity (FIG. 2).

As illustrated in Table 2 with an example set of 10 results, the method of the invention may also provide a high level of resolution up to 8 digits.

TABLE 2
Genotyping of HLA-A, -B, -C, -DRB1, -DQB1, -DQA1 and -DPB1 loci of 10
DNA samples with a level of resolution up to 8 digits.

| Name | HLA-A | HLA-A | HLA-B | HLA-B |
|---|---|---|---|---|
| 1113061961 | A*26:01:01 | A*30:01:01 | B*07:05:01 | B*13:02:01 |
| 1113062191 | A*02:01:01:01 | A*32:01:01 | B*07:02:01 | B*08:01:01 |
| 1113062201 | A*03:01:01:01 | A*25:01:01 | B*07:02:01 | B*18:01:01:02 |
| 1113062211 | A*02:01:01:01 | A*32:01:01 | B*40:01:02 | B*40:01:02 |
| 1113062221 | A*01:01:01:01 | A*02:01:01:01 | B*08:01:01 | B*27:05:02 |
| 1113062231 | A*25:01:01 | A*68:01:01:02 | B*18:01:01:02 | B*44:02:01:01 |
| 1113061161 | A*02:01:01:01 | A*24:02:01:01 | B*39:06:02:01 | B*51:01:01:01 |
| 1113062251 | A*02:01:01:01 | A*24:02:01:01 | B*35:08:01 | B*44:02:01:01 |
| 1113062591 | A*02:01:01:01 | A*03:01:01:01 | B*07:02:01 | B*44:02:01:01 |
| 1113062691 | A*01:01:01:01 | A*24:02:01:01 | B*08:01:01 | B*15:24 |

| Name | HLA-C | HLA-C | HLA-DRB1 | HLA-DRB1 |
|---|---|---|---|---|
| 1113061961 | C*06:02:01:01 | C*15:05:02 | DRB1*01:01:01 | DRB1*07:01:01:01 |
| 1113062191 | C*07:01:01:01 | C*07:02:01:03 | DRB1*15:01:01:01 | DRB1*15:01:01:01 |
| 1113062201 | C*07:02:01:03 | C*12:03:01:01 | DRB1*15:01:01:01 | DRB1*15:01:01:01 |
| 1113062211 | C*03:04:01:01 | C*03:04:01:01 | DRB1*04:05:01 | DRB1*08:22 |
| 1113062221 | C*02:02:02 | C*07:01:01:01 | DRB1*03:01:01:01 | DRB1*13:01:01 |
| 1113062231 | C*07:04:01 | C*12:03:01:01 | DRB1*01:01:01 | DRB1*15:01:01:01 |
| 1113061161 | C*05:01:01:02 | C*07:02:01:03 | DRB1*08:01:01 | DRB1*15:01:01:01 |
| 1113062251 | C*04:01:01:01 | C*05:01:01:02 | DRB1*04:01:01 | DRB1*11:04:01 |
| 1113062591 | C*05:01:01:02 | C*07:02:01:03 | DRB1*01:01:01 | DRB1*15:01:01:01 |
| 1113062691 | C*03:03:01 | C*07:01:01:01 | DRB1*04:01:01 | DRB1*11:01:01 |

| Name | HLA-DQB1 | HLA-DQB1 | HLA-DQA1 |
|---|---|---|---|
| 1113061961 | DQB1*02:02:01 | DQB1*05:01:01:01 | DQA1*01:01:01 |
| 1113062191 | DQB1*06:02:01 | DQB1*06:02:01 | DQA1*01:02:01 |
| 1113062201 | DQB1*06:02:01 | DQB1*06:02:01 | DQA1*01:02:01 |
| 1113062211 | DQB1*03:02:01 | DQB1*04:02:01 | DQA1*03:01:01 |
| 1113062221 | DQB1*02:01:01 | DQB1*06:03:01 | DQA1*01:03:01 |
| 1113062231 | DQB1*05:01:01:01 | DQB1*06:02:01 | DQA1*01:01:01 |
| 1113061161 | DQB1*04:02:01 | DQB1*06:02:01 | DQA1*01:02:01 |
| 1113062251 | DQB1*03:01:01:01 | DQB1*03:01:01:01 | DQA1*03:03:01 |
| 1113062591 | DQB1*05:01:01:01 | DQB1*06:02:01 | DQA1*01:02:01 |
| 1113062691 | DQB1*03:01:01:01 | DQB1*03:01:01:01 | DQA1*03:03:01 |

| Name | HLA-DQA1 | HLA-DPB1 | HLA-DPB1 |
|---|---|---|---|
| 1113061961 | DQA1*02:01 | DBB1*11:01:01 | DPB1*17:01 |
| 1113062191 | DQA1*04:01:02 | DBB1*03:01:01 | DPB1*04:01:01:01 |
| 1113062201 | DQA1*04:01:02 | DPB1*02:01:02 | DPB1*04:01:01:01 |
| 1113062211 | DQA1*04:01:02 | DPB1*01:01:01 | DPB1*02:01:02 |
| 1113062221 | DQA1*05:01:01 | DPB1*04:01:01:01 | DPB1*04:01:01:01 |
| 1113062231 | DQA1*04:01:02 | DPB1*04:01:01:01 | DPB1*04:01:01:01 |
| 1113061161 | DQA1*04:01:02 | DPB1*03:01:01 | DPB1*04:01:01 |
| 1113062251 | DQA1*05:05:01 | DPB1*02:01:02 | DPB1*02:01:02 |
| 1113062591 | DQA1*04:01:02 | DPB1*04:01:01:01 | DPB1*04:01:01:01 |
| 1113062691 | DQA1*05:05:01 | DPB1*04:01:01:01 | DPB1*14:01 |

All these results were concordant with previous results obtained by SBT (8K6003, 8K6103, 8K6204, 8K6303, 8K6501 reagents form Celera) and/or SSP technical approach and available for all loci excepted DQA1.

In an additional experiment, HLA-DQB1 locus of several samples was amplified using the following set of primers:

Forward primer: AGACCCAGSTACATCAGATCCATCAGGTC (SEQ ID NO: 37) corresponding to the primer CL2 F7 with four additional nucleotides at the 5′ end of SEQ ID NO: 30, and

Reverse primer: ATTATGCGTGACAGCCACTGTAGGACT (SEQ ID NO: 38) corresponding to the primer CL2 R6 with six additional nucleotides at the 5′ end of SEQ ID NO: 31.

The PCR reaction was performed in a total volume of 20 μl, containing 0.1 μL of 25 μM each primer, 2 μl of dNTP (2 mM), 80 ng DNA, 2 U long range PCR enzyme (MyFi™ DNA Polymerase, Bioline) and 1× corresponding reaction buffer.

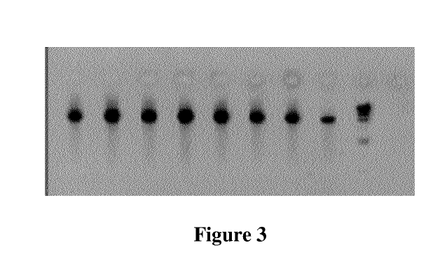

The PCR products were controlled on an electrophoresis gel (FIG. 3).

The sequences of these samples were obtained and analysed as described above, and provided a genotyping of HLA-DQB1 loci with a level of resolution up to 8 digits.

1-15. (canceled)

16. A method for determining the HLA genotype of a DNA sample, comprising:
a) contacting, in a reaction vessel, the DNA sample with an amplification reaction mixture comprising a set of amplification primers targeting HLA-A, HLA-B and HLAC genes, said primers comprising the entire sequences set forth in SEQ ID NOs: 1 to 14 or truncated forms thereof in which one to five nucleotides are missing at their 5′ termini;
b) amplifying the targeted sequences using a primer-dependent DNA amplification reaction thereby producing amplicons; and
c) determining the sequence of said amplicons.

17. The method according to claim 16, wherein step a) of the method further comprises contacting, in a distinct reaction vessel, the DNA sample with an amplification reaction mixture comprising a set of amplification primers comprising the sequences set forth in SEQ ID NOs: 19 to 24, or truncated forms thereof in which one to five nucleotides are missing at their 5′ termini, targeting HLA-DRB1, HLA-DRB3, HLA-DRB4 and HLA-DRB5 genes.

18. The method according to claim 16, wherein step a) of the method further comprises contacting, in a distinct reaction vessel, the DNA sample with an amplification reaction mixture comprising a set of amplification primers comprising the sequences set forth in SEQ ID NOs: 25 to 28, or truncated forms thereof in which one to five nucleotides are missing at their 5′ termini, targeting HLA-DPB1 gene.

19. The method according to claim 16, wherein step a) of the method further comprises contacting, in a distinct reaction vessel, the DNA sample with an amplification reaction mixture comprising a set of amplification primers comprising the sequences set forth in SEQ ID NOs: 29 to 32, or truncated forms thereof in which one to five nucleotides are missing at their 5′ termini, targeting HLA-DQB1 gene.

20. The method according to claim 16, wherein step a) of the method further comprises contacting, in a distinct reaction vessel, the DNA sample with an amplification reaction mixture comprising a set of amplification primers comprising the sequences set forth in SEQ ID NOs: 33 to 36, or truncated forms thereof in which one to five nucleotides are missing at their 5′ termini, targeting HLA-DQA1 gene.

21. The method according to claim 16, wherein step a) of the method further comprises contacting, in a distinct reaction vessel, the DNA sample with an amplification reaction mixture comprising a set of amplification primers comprising the sequences set forth in SEQ ID NOs: 13 and 14, or truncated forms thereof in which one to five nucleotides are missing at their 5′ termini, targeting HLA-A gene.

22. The method according to claim 16, wherein step a) of the method further comprises contacting, in a distinct reaction vessel, the DNA sample with an amplification reaction mixture comprising a set of amplification primers comprising the sequences set forth in SEQ ID NOs: 15 to 17, or truncated forms thereof in which one to five nucleotides are missing at their 5′ termini, targeting HLA-B gene.

23. The method according to claim 16, wherein step a) of the method further comprises contacting, in a distinct reaction vessel, the DNA sample with an amplification reaction mixture comprising a set of amplification primers comprising the sequences set forth in SEQ ID NOs: 15 and 18, or truncated forms thereof in which one to five nucleotides are missing at their 5′ termini, targeting HLA-C gene.

24. The method according to claim 16, wherein in step b), targeted sequences in each reaction vessel are amplified using a uniform thermocycling profile.

25. The method according to claim 16, wherein in step b) the annealing temperature is in the range of 55 to 65° C.

26. The method according to claim 16, wherein the primer-dependent DNA amplification reaction is a PCR reaction.

27. The method according to claim 16, wherein in step c), the sequences of amplicons are determined using a “next generation sequencing” (NGS) method.

28. The method according to claim 16, further comprising comparing the determined sequences of the amplicons with the DNA sequences of known HLA types.

29. A kit for determining the HLA genotype of a DNA sample, comprising a set of amplification primers comprising the entire sequences set forth in SEQ ID NOs: 1 to 14 or truncated forms thereof in which one to five nucleotides are missing at their 5′ termini.

30. The kit according to claim 29, further comprising:
a set of amplification primers comprising the entire sequences set forth in SEQ ID NOs: 19 to 24 or truncated forms thereof in which one to five nucleotides are missing at their 5′ termini; and/or
a set of amplification primers comprising the entire sequences set forth in SEQ ID NOs: 25 to 28 or truncated forms thereof in which one to five nucleotides are missing at their 5′ termini; and/or
a set of amplification primers comprising the entire sequences set forth in SEQ ID NOs: 29 to 32 or truncated forms thereof in which one to five nucleotides are missing at their 5′ termini; and/or
a set of amplification primers comprising the entire sequences set forth in SEQ ID NOs: 33 to 36 or truncated forms thereof in which one to five nucleotides are missing at their 5′ termini; and/or
a set of amplification primers comprising the entire sequences set forth in SEQ ID NOs: 15 to 18 or truncated forms thereof in which one to five nucleotides are missing at their 5′ termini.


SEQUENCE LISTING (38 sequences)

SEQ ID NO: 1 - DNA, 25 bp, Artificial Sequence
  Forward primer CL1_F1
cggggctctcagggtctcaggctcc

SEQ ID NO: 2 - DNA, 25 bp, Artificial Sequence
  forward primer CL1_F2
cgtggctctcagggtctcaggcccc

SEQ ID NO: 3 - DNA, 25 bp, Artificial Sequence
  Forward primer CL1_F3
ttcccactcccattgggtgtcgggt

SEQ ID NO: 4 - DNA, 25 bp, Artificial Sequence
  Forward primer CL1_F4
ttcccactcccattgggtgtcggat

SEQ ID NO: 5 - DNA, 25 bp, Artificial Sequence
  Forward primer CL1_F5
ttcccactcccattgggtattggat

SEQ ID NO: 6 - DNA, 25 bp, Artificial Sequence
  Forward primer CL1_F6
ttctcactcccattgggtgtcgggt

SEQ ID NO: 7 - DNA, 27 bp, Artificial Sequence
  Reverse primer CL1_R1
gcctttgcagaaagagatgccagaggc

SEQ ID NO: 8 - DNA, 27 bp, Artificial Sequence
  Reverse primer CL1_R2
gtgcctttgcagaaacaaagtcagggt

SEQ ID NO: 9 - DNA, 27 bp, Artificial Sequence
  Reverse primer CL1_R3
gcctttgtagaaagagatgccagaggc

SEQ ID NO: 10 - DNA, 22 bp, Artificial Sequence
  Reverse primer CL1_R4
cagtcccacacaggcagctgtc

SEQ ID NO: 11 - DNA, 22 bp, Artificial Sequence
  Reverse primer CL1_R5
agtcccacacaaggcagctgtc

SEQ ID NO: 12 - DNA, 22 bp, Artificial Sequence
  Reverse primer CL1_R6
ggtccctcacaagacagctgtc

SEQ ID NO: 13 - DNA, 24 bp, Artificial Sequence
  Forward primer CL1_F7
ctccgcagtttcttttctccctct

SEQ ID NO: 14 - DNA, 24 bp, Artificial Sequence
  Reverse primer CL1_R7
ttcaagtcacaaagggaagggcag

SEQ ID NO: 15 - DNA, 22 bp, Artificial Sequence
  Forward primer CL1_F8
ccccactcccctgagtttcact

SEQ ID NO: 16 - DNA, 20 bp, Artificial Sequence
  Reverse primer CL1_R8
catctcagtccctcacaaga

SEQ ID NO: 17 - DNA, 20 bp, Artificial Sequence
  Reverse primer CL1_R9
catctcggtccctcacaaga

SEQ ID NO: 18 - DNA, 23 bp, Artificial Sequence
  Reverse primer CL1_R10
catctcagtcccacacaggcagc

SEQ ID NO: 19 - DNA, 27 bp, Artificial Sequence
  Forward primer CL2_F1
ccagnrgastggagaggtctgttttcc

SEQ ID NO: 20 - DNA, 23 bp, Artificial Sequence
  Forward primer CL2_F2
tcactgctctttaagctccccca

SEQ ID NO: 21 - DNA, 26 bp, Artificial Sequence
  Reverse primer CL2_R1
agccacaggggaggacattttctgca

SEQ ID NO: 22 - DNA, 26 bp, Artificial Sequence
  Reverse primer CL2_R2
agccacaagggaggacattttctgca

SEQ ID NO: 23 - DNA, 26 bp, Artificial Sequence
  Reverse primer CL2_R3
agccacaaggatggacattttctgca

SEQ ID NO: 24 - DNA, 24 bp, Artificial Sequence
  Reverse primer CL2_R12
ctgaggaagccacaagggaggaca

SEQ ID NO: 25 - DNA, 23 bp, Artificial Sequence
  Forward primer CL2_F4
gatgagagtggcgcctccgctca

SEQ ID NO: 26 - DNA, 25 bp, Artificial Sequence
  Forward primer CL2_F5
cagccctgggtgggaagatttggga

SEQ ID NO: 27 - DNA, 24 bp, Artificial Sequence
  Reverse primer CL2_R4
ttcccttcctggaggagcctcagt

SEQ ID NO: 28 - DNA, 26 bp, Artificial Sequence
  Reverse primer CL2_R5
tgatctctgcttccttcagcaatgga

SEQ ID NO: 29 - DNA, 21 bp, Artificial Sequence
  Forward primer CL2_F6
cagctccagtgctgattggtt

SEQ ID NO: 30 - DNA, 25 bp, Artificial Sequence
  Forward primer CL2_F7
ccagstacatcagatccatcaggtc

SEQ ID NO: 31 - DNA, 21 bp, Artificial Sequence
  Reverse primer CL2_R6
cgtgacagccactgtaggact

SEQ ID NO: 32 - DNA, 21 bp, Artificial Sequence
  Reverse primer CL2_R7
ggggatgaaaggagatgacct

SEQ ID NO: 33 - DNA, 25 bp, Artificial Sequence
  Forward primer CL2_F8
aaggggattgcccygtctccttcca

SEQ ID NO: 34 - DNA, 26 bp, Artificial Sequence
  Forward primer CL2_F9
ggcagggtttggtttgggtgtcttca

SEQ ID NO: 35 - DNA, 25 bp, Artificial Sequence
  Reverse primer CL2_R8
gccacttcccaattcccctacaact

SEQ ID NO: 36 - DNA, 26 bp, Artificial Sequence
  Reverse primer CL2_R9
gcacctgcaacaggrcagacatgaga

SEQ ID NO: 37 - DNA, 29 bp, Artificial Sequence
  Forward primer CL2_F7 with four additional nucleotides at the 5′ end
agacccagstacatcagatccatcaggtc

SEQ ID NO: 38 - DNA, 27 bp, Artificial Sequence
  Reverse primer CL2_R6 with six additional nucleotides at the 5′ end
attatgcgtgacagccactgtaggact